(19) United States
(12) Patent Application Publication
(10) Pub. No.: US 2025/0288588 A1
(43) Pub. Date: Sep. 18, 2025

(54) SUBSTITUTED IMIDAZOPYRIDINES AND IMIDAZOPYRIMIDINES, AND USE OF SAME FOR TREATING, AMELIORATING, AND/OR PREVENTING RETINAL DEGENERATION

(71) Applicant: Yale University, New Haven, CT (US)

(72) Inventors: Mark Fields, New Haven, CT (US); Denton Hoyer, New Haven, CT (US); Lucian Del Priore, New Haven, CT (US); Huey Cai, New Haven, CT (US)

(21) Appl. No.: 18/862,727

(22) PCT Filed: May 4, 2023
(86) PCT No.: PCT/US2023/020978

§ 371 Date: Nov. 4, 2024

Related U.S. Application Data
(60) Provisional application No. 63/338,264, filed on May 4, 2022.
(60) Provisional application No. 63/411,405, filed on Sep. 29, 2022.

(51) Int. Cl. A61K 31/519; A61K 31/437; A61K 47/40; C07D 471/04; C07D 487/04

(52) U.S. Cl. CPC A61K 31/519; A61K 31/437; A61K 47/40; C07D 471/04; C07D 487/04

(57) ABSTRACT

The disclosure provides a method of treating, ameliorating, and/or preventing retinal degenerations, such as but not limited to anterior segment ocular disorders and/or age-related macular degeneration (AMD), in a subject. In certain embodiments, the method comprises administering to the subject a therapeutically effective amount of a compound of the disclosure. In certain embodiments, the compound of the disclosure prevents and/or minimizes cellular assault, such as oxidative stress-related cellular assault, and/or promote cell viability.

### CROSS-REFERENCE TO RELATED APPLICATIONS

The present application claims priority under 35 U.S.C. § 119 (e) to U.S. Provisional Patent Application No. 63/338,264 filed May 4, 2022 and U.S. Provisional Patent Application No. 63/411,405 filed Sep. 29, 2022, all of which are incorporated herein by reference in their entireties.

### BACKGROUND

Retinal degenerations are a leading cause of untreatable blindness worldwide. There are many forms of this disease, including retinitis pigmentosa (RP), which is known to be caused by around 200 different gene defects; choroideremia; retinal ganglion cell atrophy in glaucoma; and age-related macular degeneration (AMD), which is the leading cause of blindness in elderly patients, affecting more than 8 million individuals in the US alone. Given the large number of patients with retinal degenerations, and the rapid increase in the aging population, the number of patients affected by these disorders is expected to increase in the future.

Aside from vitamins and antioxidants recommended by the Age-Related Eye Disease Study, there is no effective therapy for 90% of AMD patients with “dry” or atrophic AMD. Other retinal degenerations mentioned above have no known treatments. Therapies are needed to slow or retard the development and progression of retinal disorders. As a specific example, there is a need to develop effective strategies to limit the progression of geographic atrophy (GA) and to prevent progression from dry to wet AMD. The AMD treatment market alone in the U.S., UK, Germany, France, Spain, Italy, and Japan will double in value from $5.3 billion in 2015 to $10.1 billion in 2023.

The retinal pigment epithelial (RPE) cells are vital for a proper functioning neurosensory retina. The cells make up a portion of the RPE-Bruch's membrane-choroid complex and perform critical functions for maintaining vision, including phagocytosis of photoreceptor outer segments, processing of retinoids, and polarized secretion of factors such as vascular endothelial growth factor (VEGF) and pigment epithelium-derived factor (PEDF).

Age-related changes in RPE cells are a hallmark of early AMD and contribute to pathology and visual morbidity associated with advanced AMD. Oxidative stress is a contributing factor to these changes and has been implicated in other diseases in which aging is a risk factor, including Alzheimer's disease and Parkinson's disease. In addition, Mendelian disorders, such as most forms of RP, can be accelerated by the presence of oxidative stress leading to the degeneration of the neurosensory retina, e.g. photoreceptors. In vitro and in vivo studies demonstrate that oxidative stress can accelerate cone photoreceptor death in animal models of RP.

Diseases of the anterior segment including the cornea, lens, and trabecular meshwork are leading causes of blindness worldwide. Oxidative stress and mitochondrial function have been implicated in these anterior segment diseases, including but not limited to Fuchs endothelial corneal dystrophy (FECD), cataracts, glaucoma, and keratoconus.

Age-related changes in cornea endothelial cells are a hallmark of FECD and contribute to pathology and visual morbidity. FECD is a progressive, bilateral disease characterized by a gradual loss of corneal endothelial cells (CEC). Loss of CEC impairs the ability of the cornea to maintain hydration, and results in a progressive decline in corneal transparency and hence a decline in vision. FECD is estimated to affect about 4% of the population, mostly in their forties and fifties. CEC are a highly metabolic cell type, exposure to sunlight and the lack of a significant capacity for natural regeneration of CEC make them susceptible to mitochondrial dysfunction and oxidative damage. Increased oxidative stress in the FECD cornea contributes to endothelial oxidative DNA damage, morphological modification, and CEC apoptosis.

Age-related cataracts are a leading cause of loss of vision among elderly individuals affecting approximately 46% of 180 million visually disabled people worldwide. Age-related changes in lens epithelial cells are a hallmark of cataract formation and contributes to pathology and visual morbidity. Increased oxidative stress is caused by factors such as ultraviolet light and hydrogen peroxide, and both are risk factors for cataract development. At present, the only effective treatment is extraction of cataractous lens followed by implantation of an artificial intraocular lens (IOL). However, this surgery carries some inherent risks of post-operative complications such as stimulation of chronic inflammation, cystoid macular edema, corneal edema, endophthalmitis, retinal detachment, vitreous hemorrhage, and other disorders. Moreover, inadequate surgical facilities and the high cost of artificial IOLs can be major limitations to treatment in developing countries.

Keratoconus is leading cause of corneal transplantation in younger individuals, accounting for approximately 25% of all transplants. Chronic keratocyte apoptosis, particularly of the anterior stromal keratocytes, can lead to stromal thinning in keratoconus. Oxidative stress is one of the key factors that contributes to keratoconus pathogenesis.

Oxidative stress, including oxidative damage to trabecular meshwork cells, has been implicated in the pathogenesis of glaucoma. It has been demonstrated that there is a statistically significant correlation between oxidative DNA damage and daily mean, minimum, and maximum intraocular pressure (IOP) values.

Thus, there is a need for early interventions that protect or rescue RPE as such an intervention would be beneficial and treat, ameliorate, and/or prevent disease progression. Further, there is a need for compositions and methods for treating, ameliorating, and/or preventing anterior segment disorders, such as but not limited to FECD, cataracts, glaucoma, and/or keratoconus. The present disclosure addresses this need.

### BRIEF SUMMARY

In one aspect, the present disclosure provides compounds of formula (I), and compositions comprising the same:

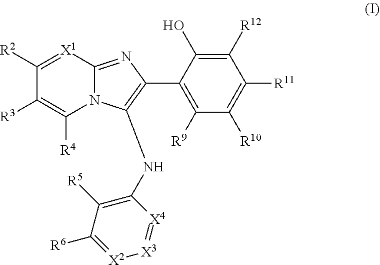

wherein variables R²-R⁶, R⁹-R¹², and X¹-X⁴ are defined elsewhere herein.

In another aspect, the present disclosure provides compounds of formula (II), and compositions comprising the same:

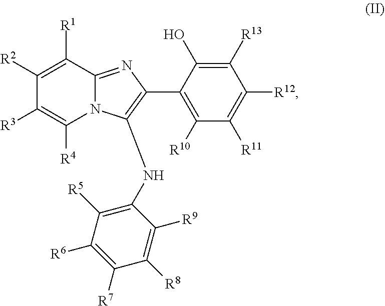

wherein variables R¹-R¹³ are defined elsewhere herein.

In yet another aspect, the present disclosure provides a method of treating, ameliorating, and/or preventing retinal degeneration in a subject. In certain embodiments, the method comprises administering to the subject a therapeutically effective amount of a compound and/or composition of the present disclosure.

In yet another aspect, the present disclosure provides a method of treating, ameliorating, and/or preventing an anterior segment ocular disorder in a subject. In certain embodiments, the method comprises administering to the subject a therapeutically effective amount of a compound and/or composition of the present disclosure.

In yet another aspect, the present disclosure provides a method of treating, ameliorating, and/or preventing cell death, and/or promoting cell viability. In certain embodiments, the method comprises administering to the subject a therapeutically effective amount of a compound and/or composition of the present disclosure.

In yet another aspect, the present disclosure provides a method of treating, ameliorating, and/or preventing blue light damage in a subject's lens epithelial cell. In certain embodiments, the method comprises administering to the subject a therapeutically effective amount of a compound and/or composition of the present disclosure.

### BRIEF DESCRIPTION OF THE DRAWINGS

The following detailed description of illustrative embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1: DH381-2 (4-bromo-2-(3-(pyridin-2-ylamino)imidazo[1,2-a]pyrimidin-2-yl)phenol) and DH421 (4-bromo-2-(3-((2-ethyl-6-methylphenyl)amino)imidazo[1,2-a]pyridin-2-yl)phenol) protected human retinal pigment epithelial (RPE) cells from oxidative stress-induced cell death. Human ARPE-19 cells (CRL-2302; American Type Culture Collection) were preincubated with 0.9 μM of DH381-2 and DH421 for 24 hours and then exposed to 300 μM tert-butyl hydroperoxide (TBHP) to induce cell death for 24 hours. DH381-2 and DH421 significantly increased cell viability of human ARPE-19 cells after exposure to TBHP. **** p<0.0001.

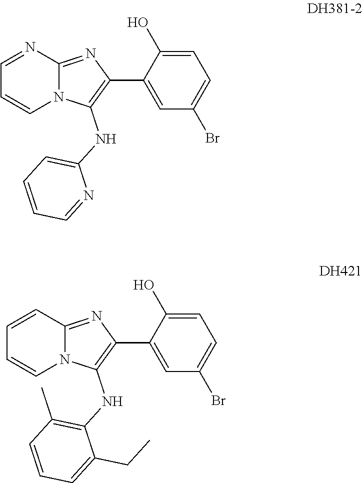 [SMILES: Oc1ccc(Br)cc1-c1nc2ncccn2c1Nc1ccccn1 ; CCc1cccc(C)c1Nc1c(-c2cc(Br)ccc2O)nc2ccccn12]

FIG. 2: DH421 protected human retinal pigment epithelial (RPE) cells from oxidative stress-induced cell death. Human ARPE-19 cells (CRL-2302; American Type Culture Collection) were preincubated with 0.9 μM of DH421 for 24 hours and then exposed to 250 μM tert-butyl hydroperoxide (TBHP) to induce cell death for 24 hours. DH421 significantly increased cell viability of human ARPE-19 cells after exposure to TBHP and has no effect on cell growth suppression. **** p<0.0001.

FIG. 3: DH421 and DH381-2 protected human retinal pigment epithelial (RPE) cells from oxidative stress-induced cell death. Human ARPE-19 cells (CRL-2302; American Type Culture Collection) were preincubated with 0.6 μM of compound DH421 or DH381-2 for 24 hours and then exposed to 300 μM tert-butyl hydroperoxide (TBHP) to induce cell death for 24 hours. Protective effects on human ARPE-19 cells treated with DH421 and DH381-2. **** p<0.0001.

FIG. 4: DH421 and DH381-2 protected human retinal pigment epithelial (RPE) cells from blue light damage. Human ARPE-19 cells (CRL-2302; American Type Culture Collection) were preincubated with 0.6 μM of compound DH421 or DH381-2 for 24 hours and then exposed to blue light (156.7 LUX for 36 hours). Protective effect of compound DH421 or DH381-2 on human ARPE-19 cells exposed to blue light (156.7 LUX for 36 hours). Control=no compound used. * p<0.05, *** p<0.01.

FIG. 5: Treatment with compound DH421 or DH381-2 enhanced human retinal pigment epithelial (RPE) cell viability on nitrite-modified extracellular matrix (ECM). Human ARPE-19 cells (CRL-2302; American Type Culture Collection) were treated with 1 μM, 3 μM, and 10 μM of compound DH421 or DH381-2 and then seeded onto nitrite-modified ECM and untreated (normal) ECM for 24 hours. Protective effects of compound DH421 or DH381-2 on human ARPE-19 cells seeded onto nitrite-modified ECM. * p<0.05, ** p<0.01.

FIG. 6: DH421 or DH381-2 protected human retinal pigment epithelial (RPE) cells from oxidative stress-induced cell death. Human RPE cells were preincubated with 0.6 μM of compound DH421 or DH381-2 for 24 hours and then exposed to 300 μM tert-butyl hydroperoxide (TBHP) to induce cell death for 24 hours. Protective effects on human RPE cells treated with DH421 and DH381-2. **** p<0.0001.

FIG. 7: DH421 and DH381-2 protected human retinal pigment epithelial (RPE) cells from blue light damage. Human RPE cells were preincubated with 0.6 μM of compound DH421 or DH381-2 for 24 hours and then exposed to blue light (156.7 LUX for 36 hours). Protective effect of compound DH421 or DH381-2 on human RPE cells exposed to blue light (156.7 LUX for 36 hours). Control=no compound. * p<0.05, *** p<0.01.

FIG. 8: Treatment with compound DH421 or DH381-2 enhanced human retinal pigment epithelial (RPE) cell viability on nitrite-modified extracellular matrix (ECM). Human RPE cells were treated with 1 μM of compound DH421 or DH381-2 and then seeded onto nitrite-modified ECM and untreated (normal) ECM for 24 hours. Protective effects of compound DH421 or DH381-2 on RPE cells seeded onto nitrite-modified ECM. * p<0.05.

FIGS. 9A-9B: DH381-2 and DH421 improved mitochondrial function after oxidative stress-induced cell death in human retinal pigment epithelial (RPE) cells. Human RPE cells were preincubated with 1 μM of compound DH381-2 and DH421 for 18 hours and then treated with 300 μM tert-butyl hydroperoxide (TBHP) for 24 hours. Oxygen consumption rate (OCR) was determined by Seahorse XF analyzer to measure mitochondrial function. ATP production was significantly reduced in TBHP-treated cells compared to control, whereas treatment with compound DH381-2 or DH421 increased ATP production when compared to TBHP-treated group. ** p<0.001.

FIG. 10: Cytotoxicity level in human retinal pigment epithelial (RPE) cells were not affected after treatment with compounds DH381-2 and DH421. Human RPE cells were preincubated with 1 μM of compound DH381-2 and DH421 for 24 hours. Cell toxicity was measured by CellTox Green Cytotoxicity Assay in human RPE cells after treatment with TBHP and compounds DH381-2 and DH421.

### DETAILED DESCRIPTION

### Definitions

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, “compound 414” or “YU162779-01” refers to ChemDiv library compound number C325-0414, or a salt and/or solvate thereof, having the formula:

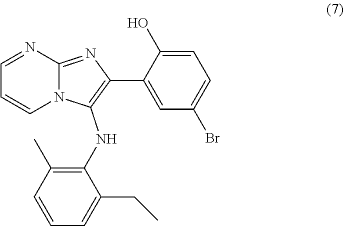 [SMILES: CCc1cccc(C)c1Nc1c(-c2cc(Br)ccc2O)nc2ncccn12]

As used herein, “compound 434” or “YU162787-01” refers to ChemDiv library compound number C325-0434, or a salt and/or solvate thereof, having the formula:

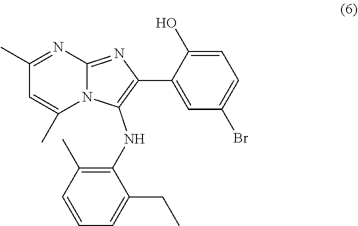 [SMILES: CCc1cccc(C)c1Nc1c(-c2cc(Br)ccc2O)nc2nc(C)cc(C)n12]

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, (including but not limited to topical, subconjunctival, subTenon's, suprachoroidal, intravitreal, or subretinal), pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the term “DH381-2” corresponds to 4-bromo-2-(3-(pyridin-2-ylamino)imidazo[1,2-a]pyrimidin-2-yl)phenol, or a salt and/or solvate thereof:

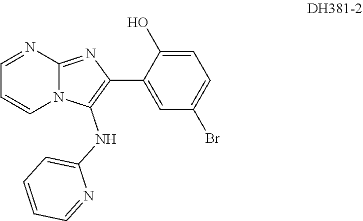 [SMILES: Oc1ccc(Br)cc1-c1nc2ncccn2c1Nc1ccccn1]

C₁₇H₁₂BrN₅O, with molecular mass of 382.21 and monoisotopic mass of 381.022. Mass spec analysis: m/z=381.

As used herein, the term “DH421” corresponds to 4-bromo-2-(3-((2-ethyl-6-methylphenyl)amino)imidazo[1,2-a]pyridin-2-yl)phenol, or a salt and/or solvate thereof:

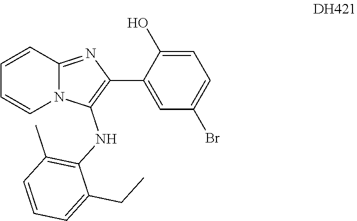 [SMILES: CCc1cccc(C)c1Nc1c(-c2cc(Br)ccc2O)nc2ccccn12]

C₂₂H₂₀BrN₃, with molecular mass of 422.318 and monoisotopic mass of 421.079. Mass spec analysis: m/z ┌M+H┐=422

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The term “independently selected from” or “independently selected from the group consisting of” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, substrate, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %. A “therapeutic” treatment is a treatment administered to a subject who exhibits signs of pathology, for the purpose of diminishing or eliminating those signs.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In its various aspects and embodiments, the disclosure provides compounds and methods for the treatment, prevention, and/or amelioration of retinal degenerations, including but not limited to AMD.

In its various aspects and embodiments, the disclosure provides compounds and methods for the treatment, prevention, and/or amelioration of anterior segment ocular disorders, such as but not limited to Fuchs endothelial corneal dystrophy (FECD), cataracts, glaucoma, and/or keratoconus.

Without wishing to be limited by theory, in certain embodiments these compounds protect retinal cells, such as RPE cells, from cell death, in a non-limiting example oxidative stress-induced cell death. In other embodiments, these compounds protect retinal cells, such as RPE cells, from cell death on damaged extracellular matrix. In yet other embodiments, these compounds increase cell viability, wherein the cell is for example a lens epithelial cell. In other embodiments, these compounds protect retinal cells, such as RPE cells, from blue light damage. Administration of compounds of the disclosure can induce expression of oxidative stress and anti-apoptotic related genes, thereby treating, ameliorating, and/or preventing retinal degenerations, including but not limited to AMD, including but not limited to “dry” AMD.

The disclosure herein should not be construed to be limited to AMD and/or oxidative stress-induced cell death. Oxidative stress is associated with a wide range of retinal degenerations, and oxidative stress has been shown to decrease photoreceptor/neuronal survival human diseases, including age-related macular degeneration (AMD), atherosclerosis, Alzheimer's disease and others. Oxidative stress is a general mechanism in which cells and tissues undergo damage in high oxygen environments. Mechanisms will vary depending on disease process, but include mitochondrial damage and dysfunction, peroxide production, free radical formation, and other mechanisms. Without loss of generality, drugs that prevent or reverse effects of tissue damage from oxidative stress are useful for slowing or reversing the progression of human disease. Non-limiting examples of such diseases include heart failure and other cardiovascular such as atherosclerosis; retinal degenerations such as age-related macular degeneration; pulmonary fibrosis; kidney (renal) disease; diabetic macular edema and retinopathies; neurodegenerations such as Alzheimer's disease; certain skeletal muscle disorders such as mitochondrial myopathy and Barth's syndrome; ocular disorders and diseases such as cataract and glaucoma; and liver disease.

As described herein, exemplary compounds of the disclosure were found to mitigate risk of oxidative damage in tissue culture models of disease. For example, compounds of the disclosure were shown to protect human retinal pigment epithelial (RPE) cells from oxidative stress-induced cell death and are beneficial to treat, ameliorate, and/or prevent diseases contemplated herein. In non-limiting embodiments, compounds of the disclosure enhance metabolic function such as adenosine triphosphate (ATP) production, basal respiration, maximal respiration, and spare respiration in RPE cells.

### Compounds

In certain embodiments, the disclosure provides a compound of formula (I), or a salt, solvate, tautomer, and/or stereoisomer thereof (such as, but not limited to, a geometric isomer and/or enantiomer and/or diastereoisomer thereof):

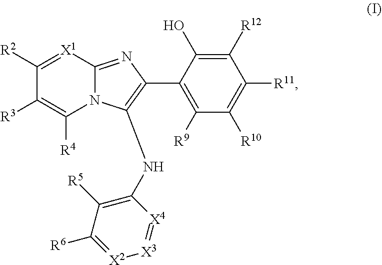

wherein:
   X¹ is N or CR¹;
   one of the following applies:
      X² is N, X³ is CR⁷, and X⁴ is CR⁸, or
      X² is CR⁷, X³ is N, and X⁴ is CR⁸, or
      X² is CR⁷, X³ is CR⁸, and X⁴ is N;
   R¹ (if present), R², R³, R⁴, R⁵, R⁶, R⁷, R⁸, R⁹, R¹⁰, R¹¹, and R¹² are independently selected from the group consisting of H, CH₃, CH₂CH₃, CH₂CH₂CH₃, CH(CH₃)₂, F, Cl, Br, and I.

In certain embodiments, X¹ is N.

In certain embodiments, X¹ is CR¹.

In certain embodiments, X² is N, X³ is CR⁷, and X⁴ is CR⁸.

In certain embodiments, X² is CR⁷, X³ is N, and X⁴ is CR⁸.

In certain embodiments, X² is CR⁷, X³ is CR⁸, and X⁴ is N.

In certain embodiments, R¹ is H. In certain embodiments, R¹ is CH₃. In certain embodiments, R¹ is CH₂CH₃. In certain embodiments, R¹ is CH₂CH₂CH₃. In certain embodiments, R¹ is CH(CH₃)₂. In certain embodiments, R¹ is F. In certain embodiments, R¹ is Cl. In certain embodiments, R¹ is Br. In certain embodiments, R¹ is I.

In certain embodiments, R² is H. In certain embodiments, R² is CH₃. In certain embodiments, R² is CH₂CH₃. In certain embodiments, R² is CH₂CH₂CH₃. In certain embodiments, R² is CH(CH₃)₂. In certain embodiments, R² is F. In certain embodiments, R² is Cl. In certain embodiments, R² is Br. In certain embodiments, R² is I.

In certain embodiments, R³ is H. In certain embodiments, R³ is CH₃. In certain embodiments, R³ is CH₂CH₃. In certain embodiments, R³ is CH₂CH₂CH₃. In certain embodiments, R³ is CH(CH₃)₂. In certain embodiments, R³ is F. In certain embodiments, R³ is Cl. In certain embodiments, R³ is Br. In certain embodiments, R³ is I.

In certain embodiments, R⁴ is H. In certain embodiments, R⁴ is CH₃. In certain embodiments, R⁴ is CH₂CH₃. In certain embodiments, R⁴ is CH₂CH₂CH₃. In certain embodiments, R⁴ is CH(CH₃)₂. In certain embodiments, R⁴ is F. In certain embodiments, R⁴ is Cl. In certain embodiments, R⁴ is Br. In certain embodiments, R⁴ is I.

In certain embodiments, R⁵ is H. In certain embodiments, R⁵ is CH₃. In certain embodiments, R⁵ is CH₂CH₃. In certain embodiments, R⁵ is CH₂CH₂CH₃. In certain embodiments, R⁵ is CH(CH₃)₂. In certain embodiments, R⁵ is F. In certain embodiments, R⁵ is Cl. In certain embodiments, R⁵ is Br. In certain embodiments, R⁵ is I.

In certain embodiments, R⁶ is H. In certain embodiments, R⁶ is CH₃. In certain embodiments, R⁶ is CH₂CH₃. In certain embodiments, R⁶ is CH₂CH₂CH₃. In certain embodiments, R⁶ is CH(CH₃)₂. In certain embodiments, R⁶ is F. In certain embodiments, R⁶ is Cl. In certain embodiments, R⁶ is Br. In certain embodiments, R⁶ is I.

In certain embodiments, R⁷ is H. In certain embodiments, R⁷ is CH₃. In certain embodiments, R⁷ is CH₂CH₃. In certain embodiments, R⁷ is CH₂CH₂CH₃. In certain embodiments, R⁷ is CH(CH₃)₂. In certain embodiments, R⁷ is F. In certain embodiments, R⁷ is Cl. In certain embodiments, R⁷ is Br. In certain embodiments, R⁷ is I.

In certain embodiments, R⁸ is H. In certain embodiments, R⁸ is CH₃. In certain embodiments, R⁸ is CH₂CH₃. In certain embodiments, R⁸ is CH₂CH₂CH₃. In certain embodiments, R⁸ is CH(CH₃)₂. In certain embodiments, R⁸ is F. In certain embodiments, R⁸ is Cl. In certain embodiments, R⁸ is Br. In certain embodiments, R⁸ is I.

In certain embodiments, R⁹ is H. In certain embodiments, R⁹ is CH₃. In certain embodiments, R⁹ is CH₂CH₃. In certain embodiments, R⁹ is CH₂CH₂CH₃. In certain embodiments, R⁹ is CH(CH₃)₂. In certain embodiments, R⁹ is F. In certain embodiments, R⁹ is Cl. In certain embodiments, R⁹ is Br. In certain embodiments, R⁹ is I.

In certain embodiments, R¹⁰ is H. In certain embodiments, R¹⁰ is CH₃. In certain embodiments, R¹⁰ is CH₂CH₃. In certain embodiments, R¹⁰ is CH₂CH₂CH₃. In certain embodiments, R¹⁰ is CH(CH₃)₂. In certain embodiments, R¹⁰ is F. In certain embodiments, R¹⁰ is Cl. In certain embodiments, R¹⁰ is Br. In certain embodiments, R¹⁰ is I.

In certain embodiments, R¹¹ is H. In certain embodiments, R¹¹ is CH₃. In certain embodiments, R¹¹ is CH₂CH₃. In certain embodiments, R¹¹ is CH₂CH₂CH₃. In certain embodiments, R¹¹ is CH(CH₃)₂. In certain embodiments, R¹¹ is F. In certain embodiments, R¹¹ is Cl. In certain embodiments, R¹¹ is Br. In certain embodiments, R¹¹ is I.

In certain embodiments, R¹² is H. In certain embodiments, R¹² is CH₃. In certain embodiments, R¹² is CH₂CH₃. In certain embodiments, R¹² is CH₂CH₂CH₃. In certain embodiments, R¹² is CH(CH₃)₂. In certain embodiments, R¹² is F. In certain embodiments, R¹² is Cl. In certain embodiments, R¹² is Br. In certain embodiments, R¹² is I.

In certain embodiments, the compound of formula (I) is a compound of formula (Ia):

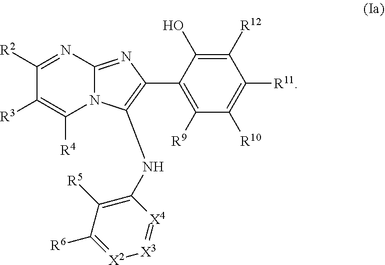

In certain embodiments, the compound of formula (I) is a compound of formula (Ib):

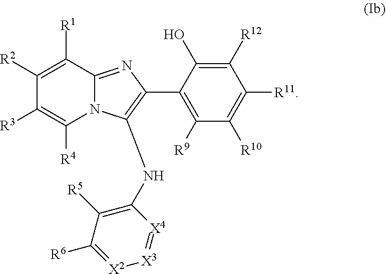

In certain embodiments, the compound of formula (I) is selected from the group consisting of:

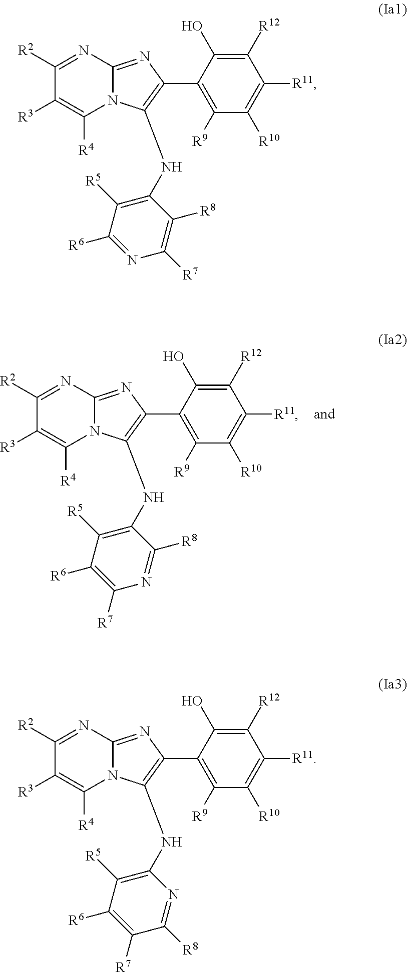

In certain embodiments, the compound of formula (I) is selected from the group consisting of:

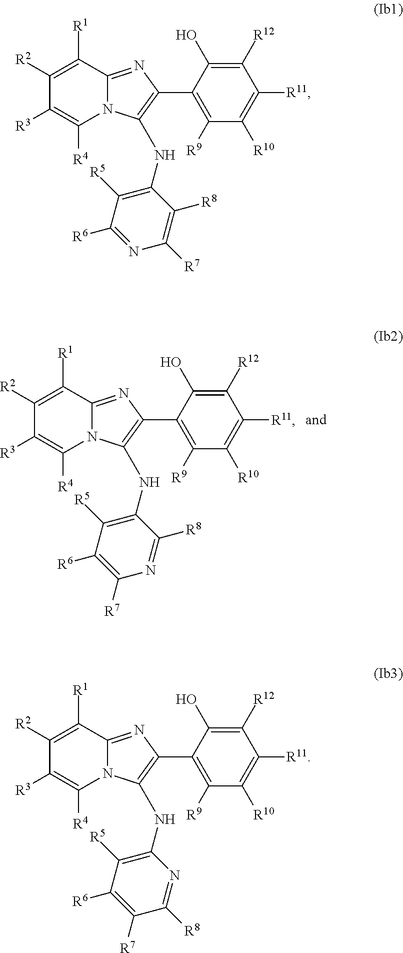

In certain embodiments, the compounds is:

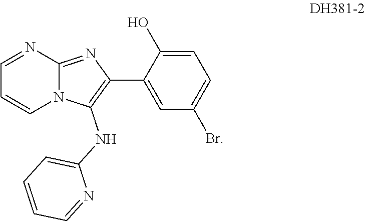 [SMILES: Oc1ccc(Br)cc1-c1nc2ncccn2c1Nc1ccccn1]

In certain embodiments, the disclosure provides a compound of formula (II), or a salt, solvate, tautomer, and/or stereoisomer (such as, but not limited to, a geometric isomer and/or enantiomer and/or diastereoisomer thereof):

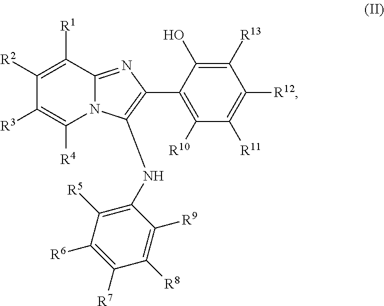

wherein:
   R¹, R², R³, R⁴, R⁶, R⁷, R⁸, R¹⁰, R¹¹, R¹², and R¹³ are independently selected from the group consisting of H, CH₃, CH₂CH₃, CH₂CH₂CH₃, CH(CH₃)₂, F, Cl, Br, and I,
   R⁵ and R⁹ are independently selected from the group consisting of CH₃, CH₂CH₃, CH₂CH₂CH₃, CH(CH₃)₂, F, Cl, Br, and I.

In certain embodiments, R¹ is H. In certain embodiments, R¹ is CH₃. In certain embodiments, R¹ is CH₂CH₃. In certain embodiments, R¹ is CH₂CH₂CH₃. In certain embodiments, R¹ is CH(CH₃)₂. In certain embodiments, R¹ is F. In certain embodiments, R¹ is Cl. In certain embodiments, R¹ is Br. In certain embodiments, R¹ is I.

In certain embodiments, R² is H. In certain embodiments, R² is CH₃. In certain embodiments, R² is CH₂CH₃. In certain embodiments, R² is CH₂CH₂CH₃. In certain embodiments, R² is CH(CH₃)₂. In certain embodiments, R² is F. In certain embodiments, R² is Cl. In certain embodiments, R² is Br. In certain embodiments, R² is I.

In certain embodiments, R³ is H. In certain embodiments, R³ is CH₃. In certain embodiments, R³ is CH₂CH₃. In certain embodiments, R³ is CH₂CH₂CH₃. In certain embodiments, R³ is CH(CH₃)₂. In certain embodiments, R³ is F. In certain embodiments, R³ is Cl. In certain embodiments, R³ is Br. In certain embodiments, R³ is I.

In certain embodiments, R⁴ is H. In certain embodiments, R⁴ is CH₃. In certain embodiments, R⁴ is CH₂CH₃. In certain embodiments, R⁴ is CH₂CH₂CH₃. In certain embodiments, R⁴ is CH(CH₃)₂. In certain embodiments, R⁷ is F. In certain embodiments, R⁴ is Cl. In certain embodiments, R⁴ is Br. In certain embodiments, R⁴ is I.

In certain embodiments, R⁵ is CH₃. In certain embodiments, R⁵ is CH₂CH₃. In certain embodiments, R⁵ is CH₂CH₂CH₃. In certain embodiments, R⁵ is CH(CH₃)₂. In certain embodiments, R⁵ is F. In certain embodiments, R⁵ is Cl. In certain embodiments, R⁵ is Br. In certain embodiments, R⁵ is I.

In certain embodiments, R⁶ is H. In certain embodiments, R⁶ is CH₃. In certain embodiments, R⁶ is CH₂CH₃. In certain embodiments, R⁶ is CH₂CH₂CH₃. In certain embodiments, R⁶ is CH(CH₃)₂. In certain embodiments, R⁶ is F. In certain embodiments, R⁶ is Cl. In certain embodiments, R⁶ is Br. In certain embodiments, R⁶ is I.

In certain embodiments, R⁷ is H. In certain embodiments, R⁷ is CH₃. In certain embodiments, R⁷ is CH₂CH₃. In certain embodiments, R⁷ is CH₂CH₂CH₃. In certain embodiments, R⁷ is CH(CH₃)₂. In certain embodiments, R⁷ is F. In certain embodiments, R⁷ is Cl. In certain embodiments, R⁷ is Br. In certain embodiments, R⁷ is I.

In certain embodiments, R⁸ is H. In certain embodiments, R⁸ is CH₃. In certain embodiments, R⁸ is CH₂CH₃. In certain embodiments, R⁸ is CH₂CH₂CH₃. In certain embodiments, R⁸ is CH(CH₃)₂. In certain embodiments, R⁸ is F. In certain embodiments, R⁸ is Cl. In certain embodiments, R⁸ is Br. In certain embodiments, R⁸ is I.

In certain embodiments, R⁹ is CH₃. In certain embodiments, R⁹ is CH₂CH₃. In certain embodiments, R⁹ is CH₂CH₂CH₃. In certain embodiments, R⁹ is CH(CH₃)₂. In certain embodiments, R⁹ is F. In certain embodiments, R⁹ is Cl. In certain embodiments, R⁹ is Br. In certain embodiments, R⁹ is I.

In certain embodiments, R¹⁰ is H. In certain embodiments, R¹⁰ is CH₃. In certain embodiments, R¹⁰ is CH₂CH₃. In certain embodiments, R¹⁰ is CH₂CH₂CH₃. In certain embodiments, R¹⁰ is CH(CH₃)₂. In certain embodiments, R¹⁰ is F. In certain embodiments, R¹⁰ is Cl. In certain embodiments, R¹⁰ is Br. In certain embodiments, R¹⁰ is I.

In certain embodiments, R¹¹ is H. In certain embodiments, R¹¹ is CH₃. In certain embodiments, R¹¹ is CH₂CH₃. In certain embodiments, R¹¹ is CH₂CH₂CH₃. In certain embodiments, R¹¹ is CH(CH₃)₂. In certain embodiments, R¹¹ is F. In certain embodiments, R¹¹ is Cl. In certain embodiments, R¹¹ is Br. In certain embodiments, R¹¹ is I.

In certain embodiments, R¹² is H. In certain embodiments, R¹² is CH₃. In certain embodiments, R¹² is CH₂CH₃. In certain embodiments, R¹² is CH₂CH₂CH₃. In certain embodiments, R¹² is CH(CH₃)₂. In certain embodiments, R¹² is F. In certain embodiments, R¹² is Cl. In certain embodiments, R¹² is Br. In certain embodiments, R¹² is I.

In certain embodiments, R¹³ is H. In certain embodiments, R¹³ is CH₃. In certain embodiments, R¹³ is CH₂CH₃. In certain embodiments, R¹³ is CH₂CH₂CH₃. In certain embodiments, R¹³ is CH(CH₃)₂. In certain embodiments, R¹³ is F. In certain embodiments, R¹³ is Cl. In certain embodiments, R¹³ is Br. In certain embodiments, R¹³ is I.

In certain embodiments, the compound of formula (II) is a compound of formula (IIa):

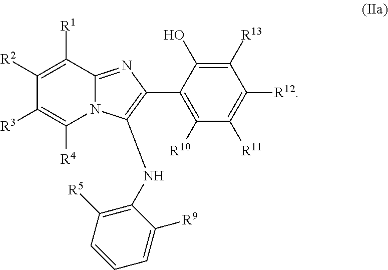

In certain embodiments, the compound of formula (II) is a compound of formula (IIb):

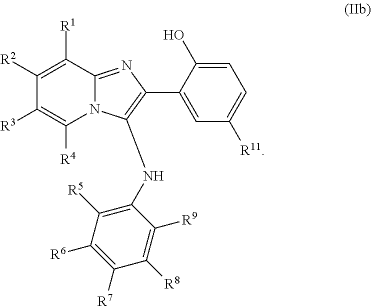

In certain embodiments, the compound of formula (II) is a compound of formula (IIc):

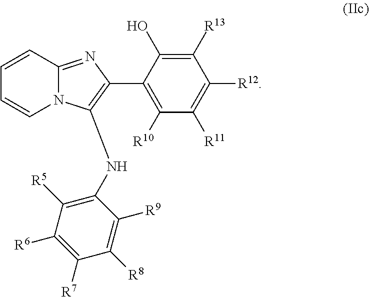

In certain embodiments, the compound of formula (II) is a compound of formula (IId):

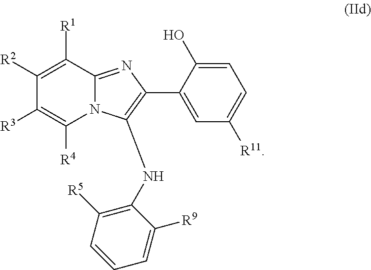

In certain embodiments, the compound of formula (II) is a compound of formula (IIe):

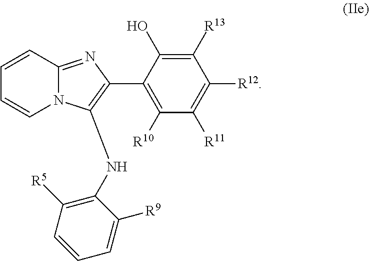

In certain embodiments, the compound of formula (II) is a compound of formula (IIf):

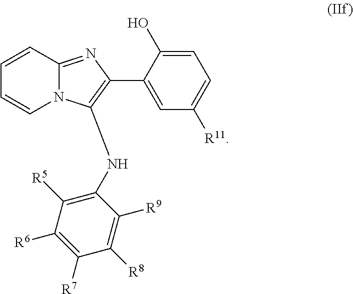

In certain embodiments, the compound of formula (II) is a compound of formula

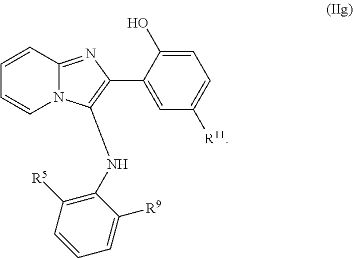

In certain embodiments, the compound of formula (II) is:

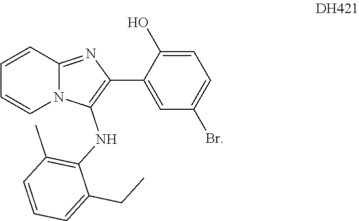 [SMILES: CCc1cccc(C)c1Nc1c(-c2cc(Br)ccc2O)nc2ccccn12]

The compounds described herein can possess one or more stereocenters, and each stereocenter can exist independently in either the (R) or(S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources or are prepared using procedures described herein.

In a non-limiting example, compounds of the disclosure can be prepared using the illustrative procedure exemplified in Example 1 herein. For example, an amine (which can be an optionally substituted 2-amino pyridine or an optionally substituted 2-amino pyrimidine, each of which can be commercially available or prepared according to methods known in the art) can be contacted with an optionally substituted 2-hydroxy benzaldehyde (which can be commercially available or prepared according to methods known in the art) in the presence of an acid (such as but not limited to formic acid, acetic acid, propionic acid, hydrochloric acid, hydrobromic acid, hydroiodic acid, sulfuric acid, phosphoric acid, and the like) or a base (such as but not limited to sodium hydroxide, potassium hydroxide, lithium hydroxide, triethylamine, Hunig's base, pyridine, and the like) in a suitable solvent (such as but not limited to methanol, ethanol, (iso) propanol, acetonitrile, tetrahydrofuran, dimethylsulfoxide, chloroform, dichloromethane, and the like) to generate the corresponding imine. The imine can be purified from the reaction mixture or used as-is in the next reaction step. The imine can then be contacted with an optionally substituted benzene isocyanide, optionally substituted 2-pyridine isocyanide, optionally substituted 3-pyridine isocyanide, or optionally substituted 4-pyridine isocyanide (each of which can be commercially available or prepared according to methods known in the art) in a suitable solvent (such as but not limited to methanol, ethanol, (iso) propanol, acetonitrile, tetrahydrofuran, dimethylsulfoxide, chloroform, dichloromethane, and the like) in the optional presence of an acid (such as but not limited to formic acid, acetic acid, propionic acid, hydrochloric acid, hydrobromic acid, hydroiodic acid, sulfuric acid, phosphoric acid, and the like) or the optional presence of a base (such as but not limited to sodium hydroxide, potassium hydroxide, lithium hydroxide, triethylamine, Hunig's base, pyridine, and the like). The desired product can be isolated from the reaction mixture by (partial) removal of solvent from the reaction mixture, addition of water and/or any other suitable solvent to the reaction mixture, seeding, or any chemical/chromatographic method known in the art.

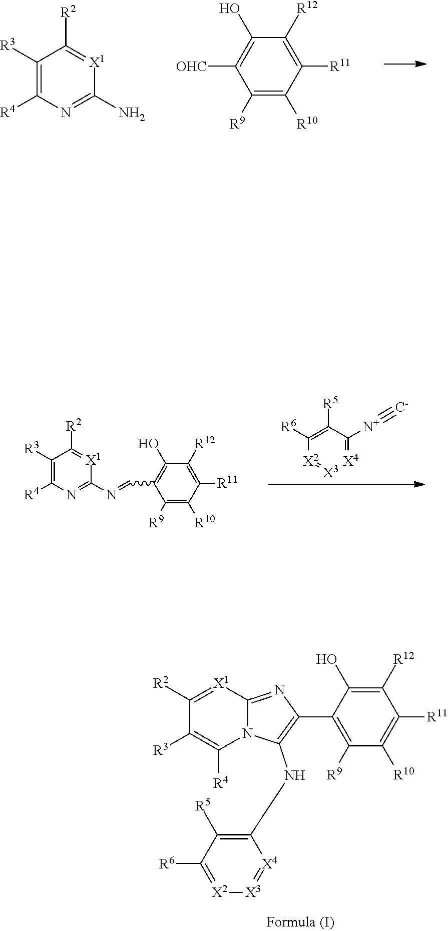

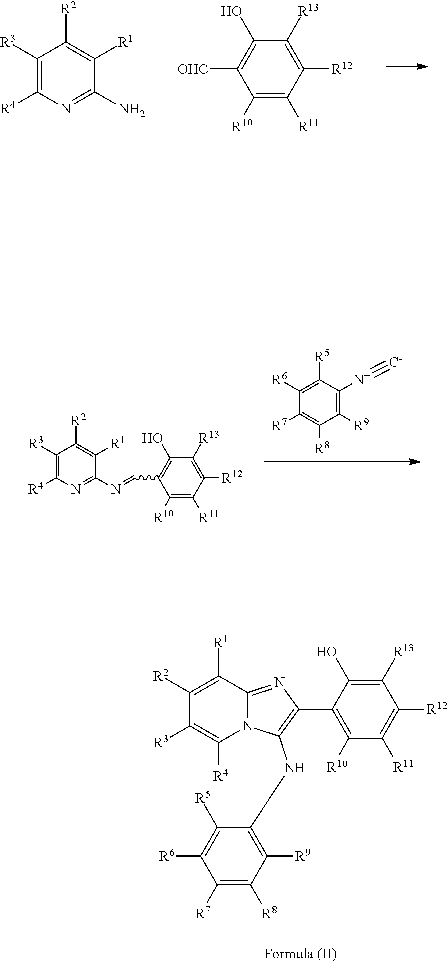

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

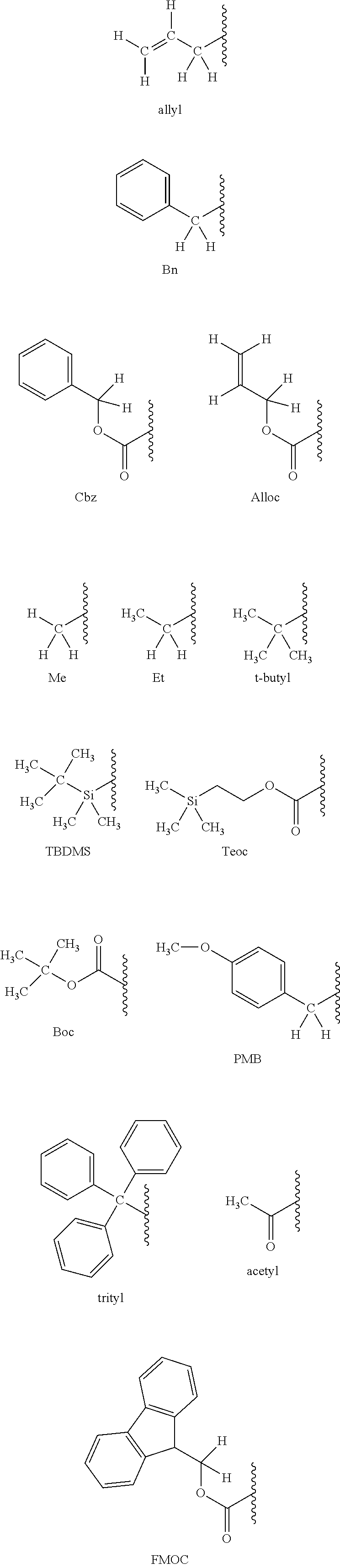

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

A skilled person will recognize that in various embodiments it can be advantageous to incorporate the compound into the pharmaceutical composition as a salt. Various counterions will be desirable for when employing different formulations and methods of administration and all pharmaceutically acceptable salts are contemplated for use with the present disclosure. In various embodiments, the pharmaceutical composition is formulated for ocular administration.

### Methods

In one aspect, the disclosure provides a method of treating, ameliorating, and/or preventing AMD (such as, but not limited to, “dry” AMD) in a subject. In certain embodiments, the method comprises administering of a subject (such as a subject in need thereof) a therapeutically effective amount of a compound contemplated herein.

In one aspect, the disclosure provides a method of treating, ameliorating, and/or preventing an anterior segment ocular disorder (such as but not limited to Fuchs endothelial corneal dystrophy (FECD), cataracts, glaucoma, and/or keratoconus). In certain embodiments, the method comprises administering of a subject (such as a subject in need thereof) a therapeutically effective amount of a compound contemplated herein.

In one aspect, the disclosure provides a method of treating, ameliorating, and/or preventing blue light damage in a subject's lens epithelial cell. In certain embodiments, the method comprises administering of a subject (such as a subject in need thereof) a therapeutically effective amount of a compound contemplated herein.

Despite advancements in the understanding of the pathophysiology of atrophic AMD, approved therapies remain elusive for this form of the disease. The atrophic or “dry” form of AMD is characterized loss of RPE cells with loss of photoreceptors and the choriocapillaris. While the etiology of the AMD is not fully understood, it is clear that risk factors such as advanced age, cigarette smoking, diet, and genetic differences (including but not limited to race) play a role in the development of the disease. RPE cells are susceptible to oxidative stress and factors such as intense illumination into the eye or toxins in cigarettes contribute to the cumulative damage caused by this process. Moreover, antioxidant capacity decreases and the efficiency of reparative systems become impaired. Age-related damage to Bruch's membrane (BM) caused by risk factors such as cigarette smoking is also associated with aberrant RPE cell behavior. These changes are a hallmark of AMD and result in retinal dysfunction and cell loss seen in atrophic AMD. The presence of hydrogen peroxide in RPE cells catalyze oxidation reactions and create reactive oxygen species (ROS) which cause irreversible damage to cells. As people age, the ability of these cells to protect against ROS is compromised. Given the observation that mitochondrial DNA damage and repair in RPE associated with aging and AMD, reducing oxidative stress is a viable therapeutic target. In certain embodiments, compounds of the disclosure prevent or minimize cell death caused by any cellular assault, which includes oxidative stress-related cellular assault or any other forms of cellular assault.

Tert-butyl hydroperoxide (TBHP) exposure has been demonstrated to disrupt junctional integrity of the RPE and cause lipid peroxidation of the membrane bilayer as well as the oxidation of glutathione, endoplasmic reticulum Ca²⁺ release, increased intracellular calcium ([Ca²⁺]), and increased mitochondrial inner membrane permeability. UV-B light damage has been shown to target mitochondrial DNA damage and produce reactive oxygen species. Chronic nitric oxide production and subsequent nitrite exposure by cigarette smoking is a risk factor strongly associated with AMD. These changes contribute to the cumulative damage to the BM and result in the age-related collagen cross linking, a decline in collagen solubility, and subsequent membrane damage. In the examples below, treatment with one or more of the compounds of the disclosure promotes cell survival as measured by a cell viability assay when challenged with tert-butyl hydroperoxide causing oxidative stress-induced cellular dysfunction and death. In certain embodiments, compounds of the disclosure can exhibit protective effects by enhancing mitochondrial respiration. In certain embodiments, enhancing metabolic activity is a valid target for degenerative diseases such as AMD.

### Administration/Dosage/Formulations

The regimen of administration may affect what constitutes an effective amount. The therapeutic formulations may be administered to the subject either prior to or after the onset of a AMD. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, preferably a mammal, more preferably a human, may be carried out using known procedures, at dosages and for periods of time effective to treat disease in the patient. An effective amount of the therapeutic compound necessary to achieve a therapeutic effect may vary according to factors such as the state of the disease or disorder in the patient; the age, sex, and weight of the patient; and the ability of the therapeutic compound to treat AMD in the patient. Dosage regimens may be adjusted to provide the optimum therapeutic response. For example, several divided doses may be administered daily, or the dose may be proportionally reduced as indicated by the exigencies of the therapeutic situation. A non-limiting example of an effective dose range for a therapeutic compound of the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of AMD in a patient.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, once every two weeks, once every three weeks, once per month, once every 2 months, once every 3 months, and/or once every 1-12 weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments thereinbetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the compounds of the disclosure can be administered ophthalmically, for example via intraocular or periocular injection. In other embodiments, the compounds are administered in a gel or a pegylated material. In other embodiments, the compounds themselves are pegylated or conjugated to a long-lasting biological molecule. In yet other embodiments, the compounds are formulated for slow delivery to the eye, for example using contact lenses comprising a polymer that releases the drug slowly, using punctual plugs, and/or using any delivery methodology that is known in the art and compatible with the present compounds.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a AMD in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual, intraocular, or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastric, intrathecal, subcutaneous, intramuscular, intradermal, intra-arterial, intravenous, intrabronchial, inhalation, topical administration, and ophthalmic (including but not limited to topical, subconjunctival, subTenon's, suprachoroidal, intravitreal, or subretinal),

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically acceptable excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated, or they may be coated by known techniques for elegance or to delay the release of the active ingredients. Formulations for oral use may also be presented as hard gelatin capsules wherein the active ingredient is mixed with an inert diluent.

The present disclosure also includes a multi-layer tablet comprising a layer providing for the delayed release of one or more compounds of the disclosure, and a further layer providing for the immediate release of a medication for treatment of certain diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular, or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Ophthalmological Administrations

The disclosure contemplates administering to the eye the compounds useful within the disclosure. Any ophthalmological formulations can be useful within the present disclosure, as well as they allow for application of the compounds useful within the disclosure to the eye.

In a non-limiting example, the compositions of the disclosure comprise gamma cyclodextrin (or γ-cyclodextrin). A solution of gamma cyclodextrin can be prepared in water at concentrations up to its solubility limit of about 23.2 mg/mL. The pH of this cyclodextrin solution can then be adjusted to a pH at which the active compound is most soluble. The active compound is then added so that the molar ratio of gamma cyclodextrin to active compound is anywhere from about 1:1 to about 10:1. The resulting suspension or solution can then be stirred for a period of time (for example, 1 hour) after which the pH is adjusted to about 5-8, preferably about 6.5-7.5. The suspension or solution can be allowed to stir for up to about 24 hours after which it is used directly, diluted with buffer to a desired concentration, and/or lyophilized to provide a powder for reconstitution. The lyophilized powder can be suspended in an amount of water that will not dissolve the powder completely but will provide a fine suspension. This suspension can then be further formulated with a thickening agent to improve adherence to the eye. Thickening agents include, but are not limited to, carboxymethylcellulose (for example, at a concentration of about 0.05-5%), or other approved agents.

### Additional Administration Forms

Additional dosage forms of this disclosure include dosage forms as described in U.S. U.S. Pat. Nos. 6,340,475; 6,488,962; 6,451,808; 5,972,389; 5,582,837; and 5,007,790. Additional dosage forms of this disclosure also include dosage forms as described in U.S. patents applications Ser. Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use within the methods of the disclosure may be administered in the form of microparticles, for example, by injection or in the form of wafers or discs by implantation.

In certain embodiments, compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, the current medical condition of the patient and the progression of AMD in the patient being treated. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### EXPERIMENTAL EXAMPLES

The disclosure is further described in detail by reference to the following experimental examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples thus specifically point out the preferred embodiments of the present disclosure, and are not to be construed as limiting in any way the remainder of the disclosure. The materials and methods employed in practicing the following examples are here described:

### Human Retinal Pigment Epithelial (RPE) Cell Culture

Immortalized human RPE cells (ARPE-19) obtained from the American Type Culture Collection (ATCC, Manassas, VA, USA) were cultured in Dulbecco's modified Eagle's medium (DMEM; Thermo Fisher Scientific, Waltham, MA) containing 10% fetal bovine serum (FBS), 100 IU/mL penicillin, 100 μg/mL streptomycin, 100 μg/mL gentamicin, and 2.5 μg/mL amphotericin B (Thermo Fisher Scientific).

### Induction of Oxidative Stress Using Tert-Butyl Hydroperoxide

Human ARPE-19 cells were plated in 96-well plates for 24 hours in DMEM supplemented with FBS and antibiotics. ARPE-19 cells were preincubated with either ciclopirox olamine (Sigma), compound 414 (Chemical Diversity, San Diego, CA), compound 434 (Chemical Diversity) or no compound for 24 hours and then exposed to varying concentrations of tert-butyl hydroperoxide (TBHP; Sigma-Aldrich, St. Louis, MO) the next day for 24 hours. Cell viability was measured the following day by RealTime-Glo™ MT cell viability assay (Promega, Madison, WI) using a BioTek FLx800™ fluorescence reader (BioTek, Winooski, VT).

### Nitrite Modification of the Extracellular Matrix (ECM)

Immortalized human RPE (ARPE-19) cells were obtained from the ATCC, cultured, and maintained in DMEM (Invitrogen-Gibco, Life Technologies) containing 10% FBS, 100 IU/mL penicillin, 100 μg/mL streptomycin, and 100 μg/mL gentamicin (Invitrogen-Gibco, Life Technologies). These cells were incubated in a humidified atmosphere of 5% CO₂ and 95% air at 37° C. ARPE-19 cells were grown on 24-well Transwell® permeable supports (Corning, Inc.) in 12-well plates or flat bottom 24-well plates for 6-8 weeks to allow the ECM to form. ARPE-19 cells were then removed by the addition of 20 mM ammonium hydroxide buffer for 20 min, and the ECM was washed with phosphate buffered saline (PBS). PBS was removed from the RPE-ECM plates and dried. Subsequently, 100 mM sodium nitrite was added to the ECM and incubated at 37° C. for 7 days. Plates were then washed with PBS and incubated with PBS for 4 hours. Finally, plates were washed with PBS to completely remove the nitrite. Cells are preincubated with drug for 24 hours and then seeded on nitrite-modified ECM for 24 hours. Cell viability was measured using RealTime-Glo MT assays (Promega) with a BioTek FLx800 fluorescence reader (Bio Tek, Winooski, VT).

### Induction of Oxidative Stress Using Blue Light Damage

Human ARPE-19 cells were plated in 96-well plates for 24 hours in DMEM supplemented with FBS and antibiotics. The cells were preincubated with drug for 24 hours and then exposed to blue light (156.7 LUX for 36 hours). Cell viability was measured after 36 hours of blue light exposure using RealTime-Glo MT assays (Promega) with a BioTek FLx800 fluorescence reader (Bio Tek, Winooski, VT).

### Statistical Analysis

All experiments were conducted at least three times with triplicates. Independent, two-tailed t tests were performed using Prism (GraphPad Software, Inc., La Jolla, CA). A criterion of α=0.05 was adopted.

### Example 1: Chemical Synthesis

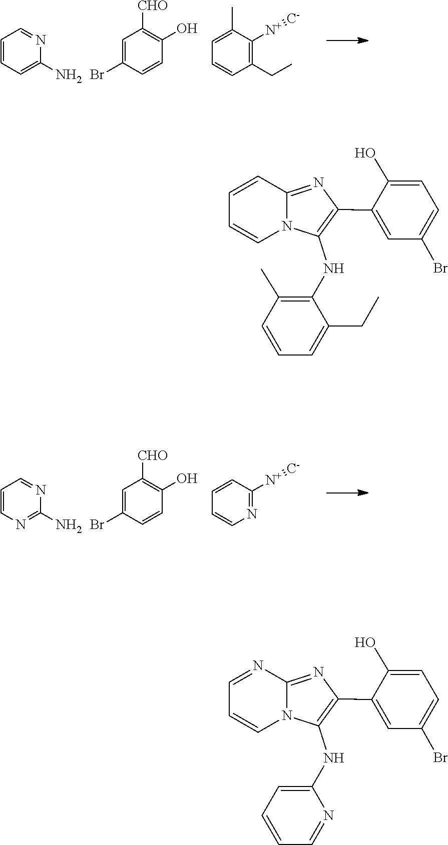

### General Methods

The solvents were purified according to the standard procedures. All other starting materials were purchased from commercial sources. Analytical TLC was performed using Polychrom SI F254 plates. Column chromatography was performed using Kieselgel Merck 60 (230-400 mesh) as the stationary phase. ¹H NMR spectra were recorded on a Varian Gemini 2000 spectrometer. Tetramethylsilane was used as internal standard. Mass spectra were recorded on an Agilent 1100 LCMSD SL instrument [electrospray ionization (ESI)].

### General Procedures

A solution of amine (2.0 mmol), aldehyde (2.0 mmol), and acetic acid (4.0 mmol) in 20 mL of methanol was stirred for 20 min at 20° C. The reaction mixture was cooled and at 0° C. and isocyanide (2.0 mmol) was added in one portion. Stirring was continued at 20° C. for additional 5-6 hours (TCL monitoring). The products crystallized from the reaction mixture or on adding several drops of water. The product was collected by filtration, washed with water (3-10 mL), dried, and washed again with hexane (50 mL). The crude product was crystallized (methanol) or purified by column chromatography (CH₂Cl₂—CH₃OH, 15:5) to give crystalline compounds.

4-Bromo-2-[3-(2-ethyl-6-methyl-anilino)imidazo[1,2-a]pyridin-2-yl]phenol (DH421). Brown powder. Yield 73%. 1H NMR (400 MHz, DMSO-d₆) δ 13.21 (d, J=3.4 Hz, 1H), 7.99 (d, J=6.6 Hz, 1H), 7.87 (t, J=3.0 Hz, 1H), 7.70 (dd, J=9.1, 3.3 Hz, 1H), 7.39 (t, J=7.9 Hz, 1H), 7.32 (d, J=3.5 Hz, 1H), 7.24 (dd, J=8.6, 2.8 Hz, 1H), 7.09-6.97 (m, 2H), 6.90 (d, J=7.4 Hz, 1H), 6.86-6.75 (m, 2H), 1.71 (d, J=3.4 Hz, 3H), 1.02 (t, J=7.4 Hz, 3H).

ESIMS. m/z: 423 (M+1)+.

4-bromo-2-(3-(pyridin-2-ylamino)imidazo[1,2-a]pyrimidin-2-yl)phenol (DH381-2). Brown powder. Yield 15%, 1H NMR (500 MHz, DMSO-d₆) δ 13.20 (s, 1H), 9.04 (s, 1H), 8.02 (t, J=5.0 Hz, 2H), 7.96 (d, J=5.0 Hz, 1H), 7.74 (dd, J=9.1, 3.6 Hz, 1H), 7.65-7.56 (m, 1H), 7.44 (dd, J=9.1, 6.5 Hz, 1H), 7.29 (dd, J=8.6, 2.6 Hz, 1H), 7.04 (q, J=6.8, 5.2 Hz, 1H), 6.87 (dd, J=8.7, 3.3 Hz, 1H), 6.79-6.70 (m, 2H).

ESIMS. m/z: 383 (M+1)+.

### Example 2

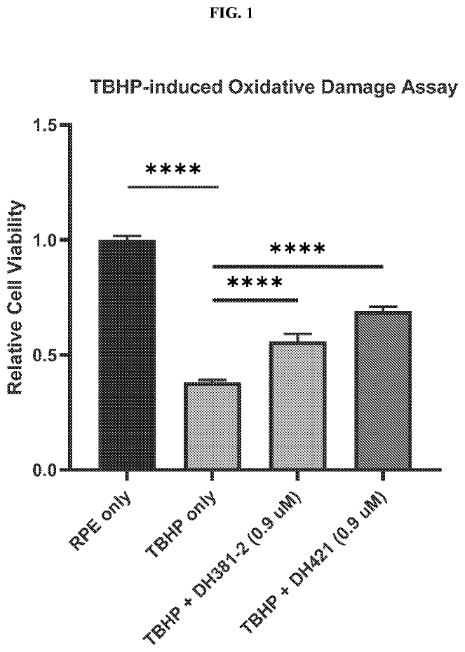

As illustrated in FIG. 1, both DH381-2 (4-bromo-2-(3-(pyridin-2-ylamino)imidazo[1,2-a]pyrimidin-2-yl)phenol) and DH421 (4-bromo-2-(3-((2-ethyl-6-methylphenyl)amino)imidazo[1,2-a]pyridin-2-yl)phenol) protected human retinal pigment epithelial (RPE) cells from oxidative stress-induced cell death. Human ARPE-19 cells (CRL-2302; American Type Culture Collection) were preincubated with 0.9 μM of DH381-2 and DH421 for 24 hours and then exposed to 300 μM tert-butyl hydroperoxide (TBHP) to induce cell death for 24 hours. DH381-2 and DH421 significantly increased cell viability of human ARPE-19 cells after exposure to TBHP. **** p<0.0001.

### Example 3

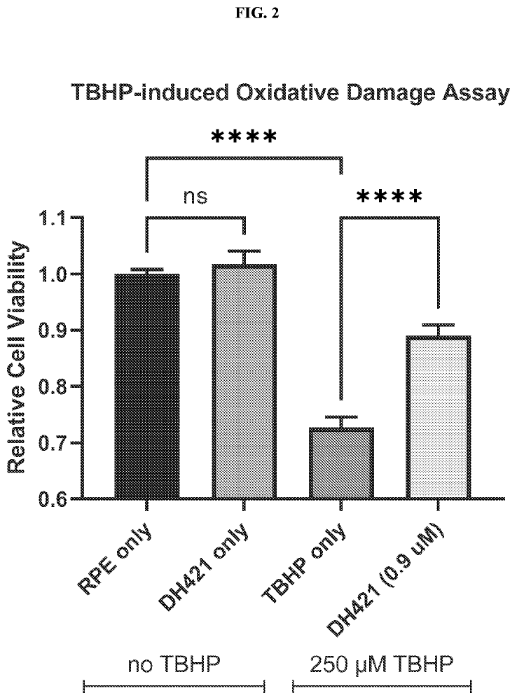

As illustrated in FIG. 2, DH421 protected human retinal pigment epithelial (RPE) cells from oxidative stress-induced cell death. Human ARPE-19 cells (CRL-2302; American Type Culture Collection) were preincubated with 0.9 μM of DH421 for 24 hours and then exposed to 250 μM tert-butyl hydroperoxide (TBHP) to induce cell death for 24 hours. DH421 significantly increased cell viability of human ARPE-19 cells after exposure to TBHP and has no effect on cell growth suppression. **** p<0.0001.

### Example 4

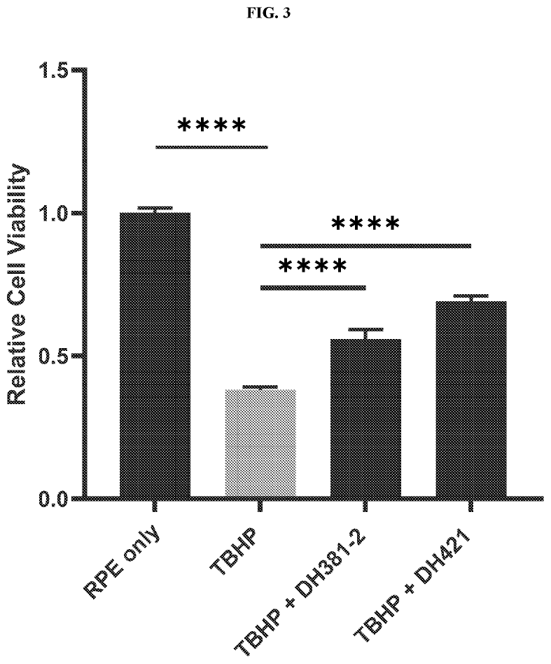

As illustrated in FIG. 3, both DH421 and DH381-2 protected human retinal pigment epithelial (RPE) cells from oxidative stress-induced cell death. Human ARPE-19 cells (CRL-2302; American Type Culture Collection) with preincubated with 0.6 μM of compound DH421 or DH381-2 for 24 hours and then exposed to 300 μM tert-butyl hydroperoxide (TBHP) to induce cell death for 24 hours. Protective effects on human ARPE-19 cells treated with DH421 and DH381-2. **** p<0.0001.

### Example 5

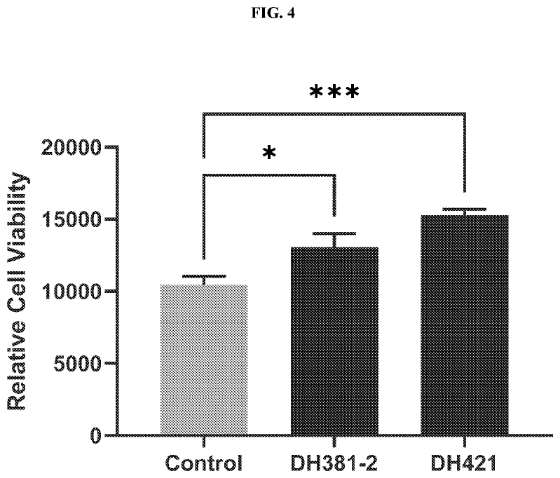

As illustrated in FIG. 4, both DH421 and DH381-2 protected human retinal pigment epithelial (RPE) cells from blue light damage. Human ARPE-19 cells (CRL-2302; American Type Culture Collection) were preincubated with 0.6 μM of compound DH421 or DH381-2 for 24 hours and then exposed to blue light (156.7 LUX for 36 hours). Protective effect of compound DH421 or DH381-2 on human ARPE-19 cells exposed to blue light (156.7 LUX for 36 hours). * p<0.05, *** p<0.01.

### Example 6

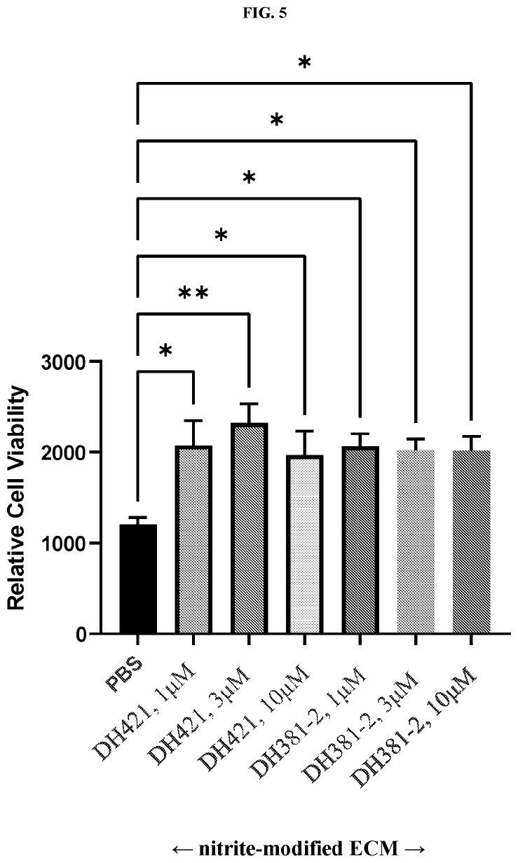

As illustrated in FIG. 5, treatment with compound DH421 or DH381-2 enhanced human retinal pigment epithelial (RPE) cell viability on nitrite-modified extracellular matrix (ECM). Human ARPE-19 cells (CRL-2302; American Type Culture Collection) were treated with 1 μM, 3 μM, and 10 μM of compound DH421 or DH381-2 and then seeded onto nitrite-modified ECM and untreated (normal) ECM for 24 hours. Protective effects of compound DH421 or DH381-2 on human ARPE-19 cells seeded onto nitrite-modified ECM. * p<0.05, ** p<0.01.

### Example 7

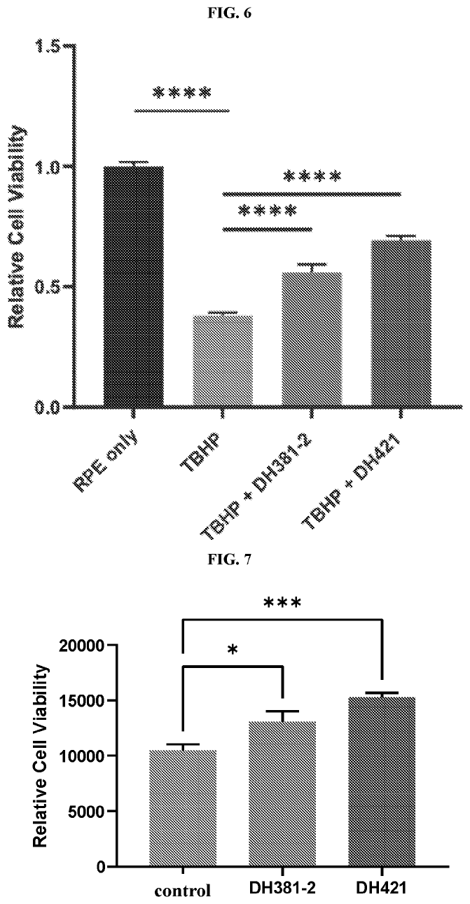

As illustrated in FIG. 6, both DH421 and DH381-2 protected human retinal pigment epithelial (RPE) cells from oxidative stress-induced cell death. Human RPE cells were preincubated with 0.6 μM of compound DH421 or DH381-2 for 24 hours and then exposed to 300 μM tert-butyl hydroperoxide (TBHP) to induce cell death for 24 hours. Protective effects on human RPE cells treated with DH421 and DH381-2. **** p<0.0001.

### Example 8

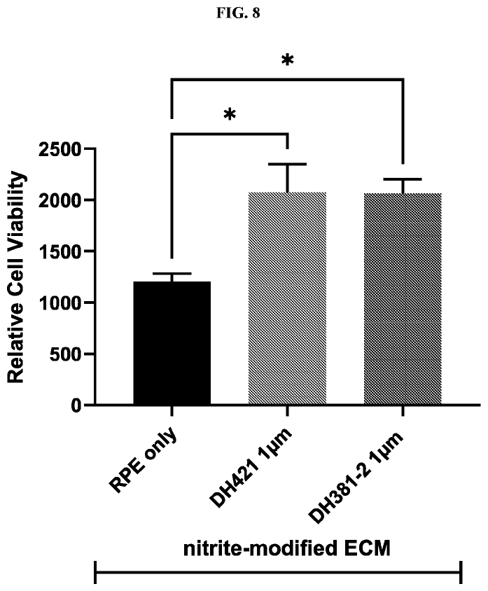

As illustrated in FIG. 7, both DH421 and DH381-2 protected human retinal pigment epithelial (RPE) cells from blue light damage. Human RPE cells were preincubated with 0.6 μM of compound DH421 or DH381-2 for 24 hours and then exposed to blue light (156.7 LUX for 36 hours). Protective effect of compound DH421 or DH381-2 on human RPE cells exposed to blue light (156.7 LUX for 36 hours). * p<0.05, *** p<0.01.

### Example 9

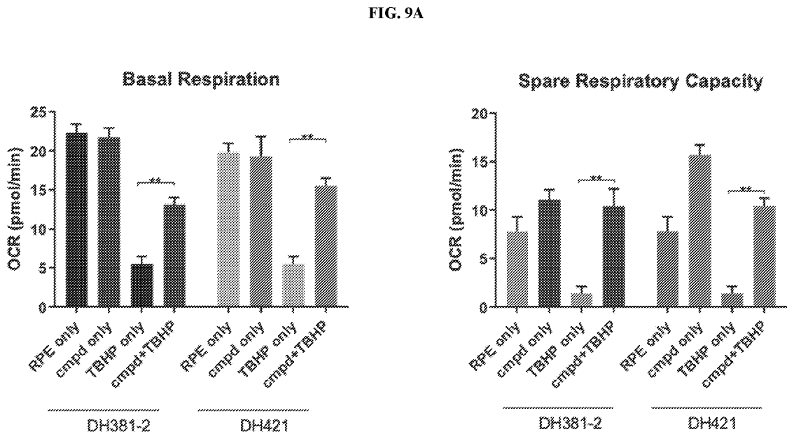

As illustrated in FIG. 8, treatment with compound DH421 or DH381-2 enhanced human retinal pigment epithelial (RPE) cell viability on nitrite-modified extracellular matrix (ECM). Human RPE cells were treated with 1 μM of compound DH421 or DH381-2 and then seeded onto nitrite-modified ECM and untreated (normal) ECM for 24 hours. Protective effects of compound DH421 or DH381-2 on RPE cells seeded onto nitrite-modified ECM. *p<0.05.

### Example 10

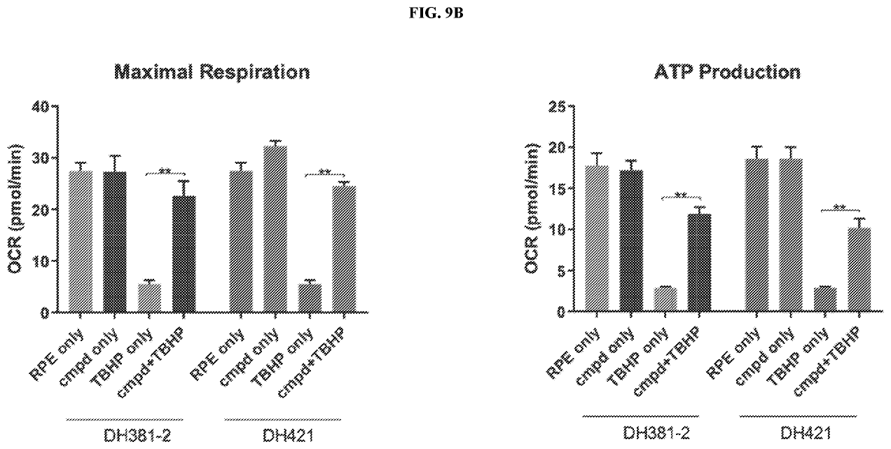

As illustrated in FIGS. 9A-9B, both DH381-2 and DH421 improved mitochondrial function after oxidative stress-induced cell death in human retinal pigment epithelial (RPE) cells. Human RPE cells were preincubated with 1 μM of compound DH381-2 and DH421 for 18 hours and then treated with 300 μM tert-butyl hydroperoxide (TBHP) for 24 hours. Oxygen consumption rate (OCR) was determined by Seahorse XF analyzer to measure mitochondrial function. ATP production was significantly reduced in TBHP-treated cells compared to control, whereas treatment with compound DH381-2 or DH421 increased ATP production when compared to TBHP-treated group. ** p<0.001.

### Example 11

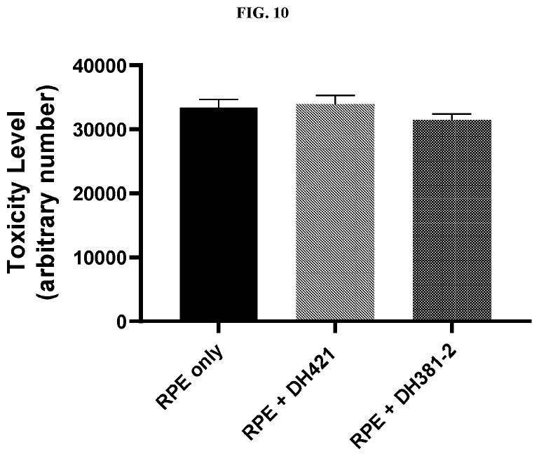

As illustrated in FIG. 10, cytotoxicity level in human retinal pigment epithelial (RPE) cells was not affected after treatment with compounds DH381-2 and DH421. Human RPE cells were preincubated with 1 μM of compound DH381-2 and DH421 for 24 hours. Cell toxicity was measured by CellTox Green Cytotoxicity Assay in human RPE cells after treatment with TBHP and compounds DH381-2 and DH421.

### Enumerated Embodiments

The following exemplary embodiments are provided, the numbering of which is not to be construed as designating levels of importance:

Embodiment 1: A compound of formula (I):

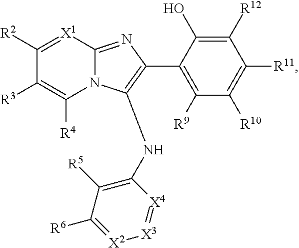

(I), wherein:
   X¹ is N or CR¹;
   one of the following applies:
      X² is N, X³ is CR⁷, and X⁴ is CR⁸, or
      X² is CR⁷, X³ is N, and X⁴ is CR⁸, or
      X² is CR⁷, X³ is CR⁸, and X⁴ is N;
   R¹ (if present), R², R³, R⁴, R⁵, R⁶, R⁷, R⁸, R⁹, R¹⁰, R¹¹, and R¹² are independently selected from the group consisting of H, CH₃, CH₂CH₃, CH₂CH₂CH₃, CH(CH₃)₂, F, Cl, Br, and I;
or a salt, solvate, tautomer, and/or stereoisomer thereof.

Embodiment 2: The compound of claim 1, wherein X¹ is N.

Embodiment 3: The compound of claim 1, wherein X¹ is CR¹.

Embodiment 4: The compound of any one of claims 1-3, wherein X² is N, X³ is CR⁷, and X⁴ is CR⁸.

Embodiment 5: The compound of any one of claims 1-3, wherein X² is CR⁷, X³ is N, and X⁴ is CR⁸.

Embodiment 6: The compound of any one of claims 1-3, wherein X² is CR⁷, X³ is CR⁸, and X⁴ is N.

Embodiment 7: The compound of any one of claims 1-6, wherein R¹⁰ is Br.

Embodiment 8: The compound of any one of claims 1-7, wherein R⁵, R⁶, R⁷, and R⁸ are H.

Embodiment 9: The compound of any one of claims 1-8, which is a compound of formula (Ia):

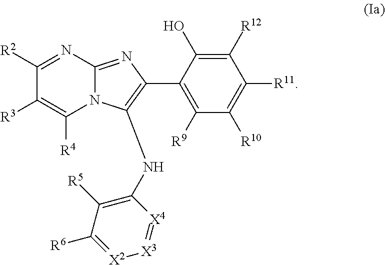

Embodiment 10: The compound of any one of claims 1-8, which is a compound of formula (Ib):

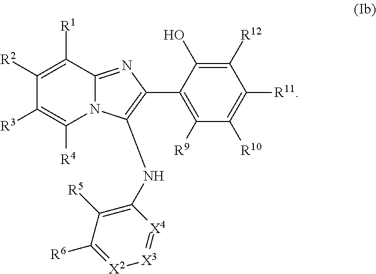

Embodiment 11: The compound of any one of claims 1-9, which is selected from the group consisting of:

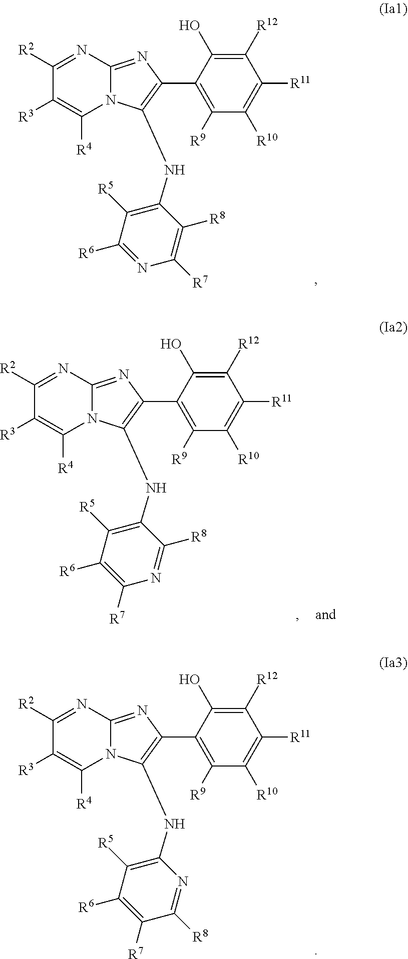

Embodiment 12: The compound of any one of claims 1-8 and 10, which is selected from the group consisting of:

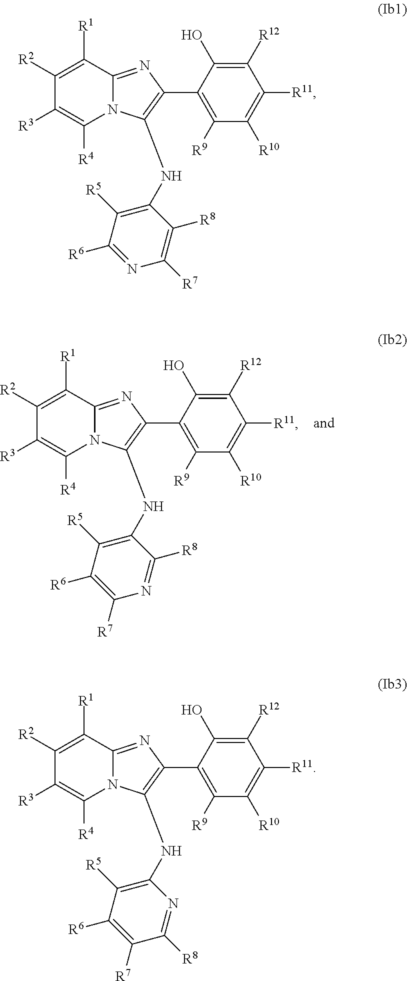

Embodiment 13: The compound of any one of claim 1-12, which is:

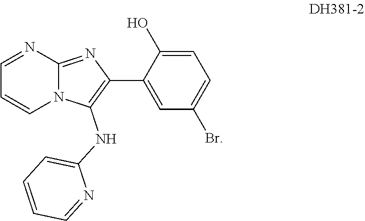 [SMILES: Oc1ccc(Br)cc1-c1nc2ncccn2c1Nc1ccccn1]

Embodiment 14: A compound of formula (II):

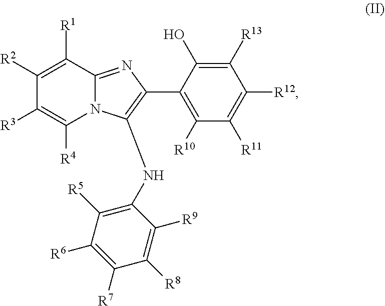

wherein:
R¹, R², R³, R⁴, R⁶, R⁷, R⁸, R¹⁰, R¹¹, R¹², and R¹³ are independently selected from the group consisting of H, CH₃, CH₂CH₃, CH₂CH₂CH₃, CH(CH₃)₂, F, Cl, Br, and I,
R⁵ and R⁹ are independently selected from the group consisting of CH₃, CH₂CH₃, CH₂CH₂CH₃, CH(CH₃)₂, F, Cl, Br, and I;
or a salt, solvate, tautomer, and/or stereoisomer thereof.

Embodiment 15: The compound of claim 14, wherein R¹¹ is Br.

Embodiment 16: The compound of any one of claims 14-15, wherein R⁶, R⁷, and R⁸ are H.

Embodiment 17: The compound of any one of claims 14-16, which is selected from the group consisting of:

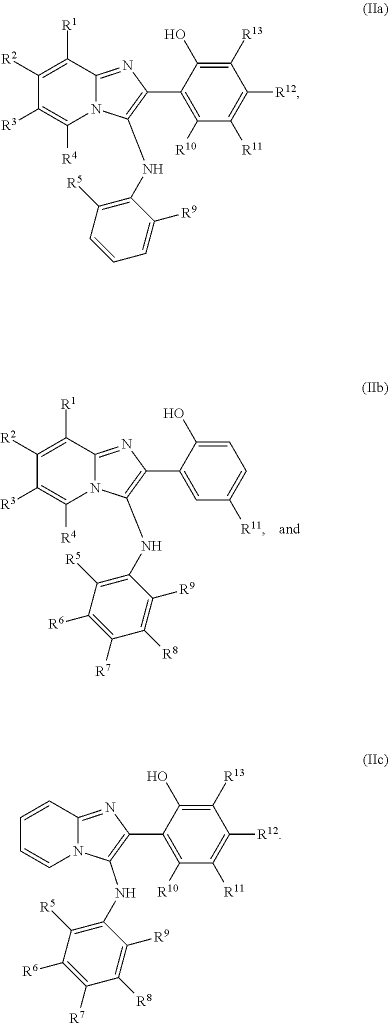

Embodiment 18: The compound of any one of claims 14-17, which is selected from the group consisting of:

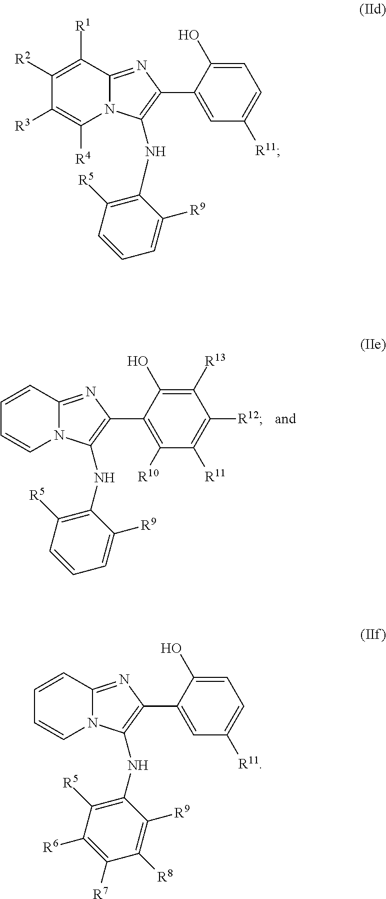

Embodiment 19: The compound of any one of claims 14-18, which is:

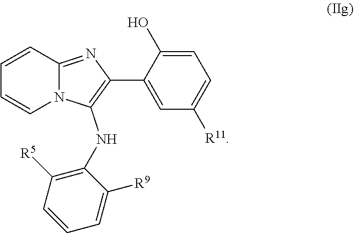

Embodiment 20: The compound of any one of claims 14-19, which is:

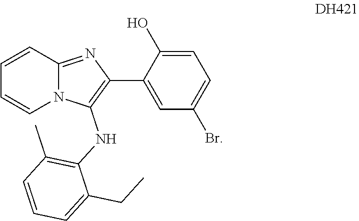 [SMILES: CCc1cccc(C)c1Nc1c(-c2cc(Br)ccc2O)nc2ccccn12]

Embodiment 21: A method of treating, ameliorating, and/or preventing retinal degeneration in a subject, the method comprising administering to the subject a therapeutically effective amount of a compound of any one of claims 1-20.

Embodiment 22: The method of claim 21, wherein the retinal degeneration comprises age-related macular degeneration (AMD).

Embodiment 23: The method of claim 21, wherein the retinal degeneration comprises ‘dry’ AMD.

Embodiment 24: A method of treating, ameliorating, and/or preventing an anterior segment ocular disorder in a subject, the method comprising administering to the subject a therapeutically effective amount of a compound of any one of claims 1-20.

Embodiment 25: The method of claim 24, wherein the disorder comprises at least one selected from the group consisting of Fuchs Endothelial Corneal Dystrophy, cataracts, glaucoma, and keratoconus.

Embodiment 26: A method of treating, ameliorating, and/or preventing cell death, and/or promoting cell viability, in a subject, the method comprising administering to the subject a therapeutically effective amount of a compound of any one of claims 1-20.

Embodiment 27: The method of claim 26, wherein the cell death comprises oxidative stress-induced cell death.

Embodiment 28: The method of claim 26, wherein the cell comprises a lens epithelial cell.

Embodiment 29: The method of claim 26, wherein the cell death is associated with at least one disease selected from the group consisting of heart failure and other cardiovascular; pulmonary fibrosis, kidney disease, diabetic macular edema and retinopathies, neurodegeneration, mitochondrial myopathy, Barth's syndrome, and liver disease.

Embodiment 30: A method of treating, ameliorating, and/or preventing blue light damage in a subject's lens epithelial cell, the method comprising administering to the subject a therapeutically effective amount of a compound of any one of claims 1-20.

Embodiment 31: The method of any one of claims 21-30, wherein the compound is formulated in a pharmaceutically acceptable composition further comprising at least one pharmaceutically acceptable excipient.

Embodiment 32: The method of any of claims 21-28 and 30-31, wherein the compound is administered ocularly.

Embodiment 33: The method of any one of claims 21-32, wherein the compound is one of the following:

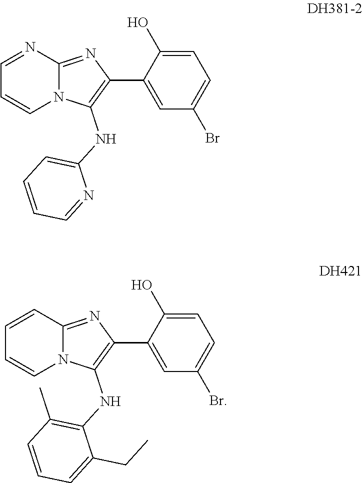 [SMILES: Oc1ccc(Br)cc1-c1nc2ncccn2c1Nc1ccccn1 ; CCc1cccc(C)c1Nc1c(-c2cc(Br)ccc2O)nc2ccccn12]

Embodiment 34: A pharmaceutical composition comprising a compound of any one of claims 1-20 and at least one pharmaceutically acceptable excipient.

Embodiment 35: The pharmaceutical composition of claim 34, which is formulated for ocular administration.

Embodiment 36; The pharmaceutical composition of any one of claims 34-35, which comprises gamma cyclodextrin (γ-cyclodextrin).

Embodiment 37: The pharmaceutical composition of any one of claims 34-36, which has pH of about 5-8.

Embodiment 38: The pharmaceutical composition of any one of claims 34-37, which is lyophilized.

Embodiment 39: The pharmaceutical composition of any one of claims 34-38, which further comprises a thickening agent.

The disclosures of each and every patent, patent application, and publication cited herein are hereby incorporated herein by reference in their entirety.

While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A compound of formula (I): 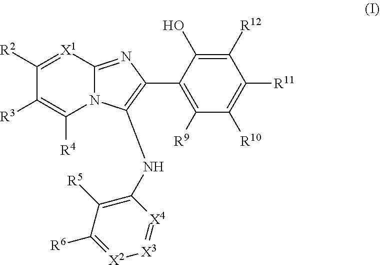
wherein:
X¹ is N or CR¹;
one of the following applies:
X² is N, X³ is CR⁷, and X⁴ is CR⁸, or
X² is CR⁷, X³ is N, and X⁴ is CR⁸, or
X² is CR⁷, X³ is CR⁸, and X⁴ is N;
R¹ (if present), R², R³, R⁴, R⁵, R⁶, R⁷, R⁸, R⁹, R¹⁰, R¹¹, and R¹² are independently selected from the group consisting of H, CH₃, CH₂CH₃, CH₂CH₂CH₃, CH(CH₃)₂, F, Cl, Br, and I;
or a salt, solvate, tautomer, or stereoisomer thereof.

2. The compound of claim 1, wherein X¹ is N.

3. The compound of claim 1, wherein X¹ is CR¹.

4. The compound of claim 1, wherein X² is N, X³ is CR⁷, and X⁴ is CR⁸.

5. The compound of claim 1, wherein X² is CR⁷, X³ is N, and X⁴ is CR⁸.

6. The compound of claim 1, wherein X² is CR⁷, X³ is CR⁸, and X⁴ is N.

7. The compound of claim 1, wherein at least one of the following applies:
R¹⁰ is Br;
R⁵, R⁶, R⁷, and R⁸ are H.

8. (canceled)

9. The compound of claim 1,
which is a compound of formula (Ia): 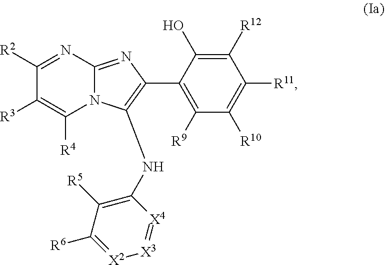
or which is a compound of formula (Ib):

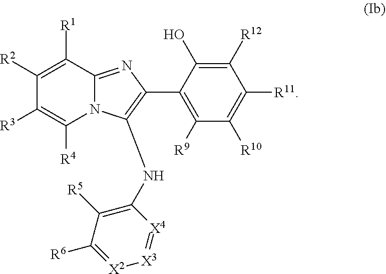

10. (canceled)

11. The compound of claim 1, which is selected from the group consisting of:

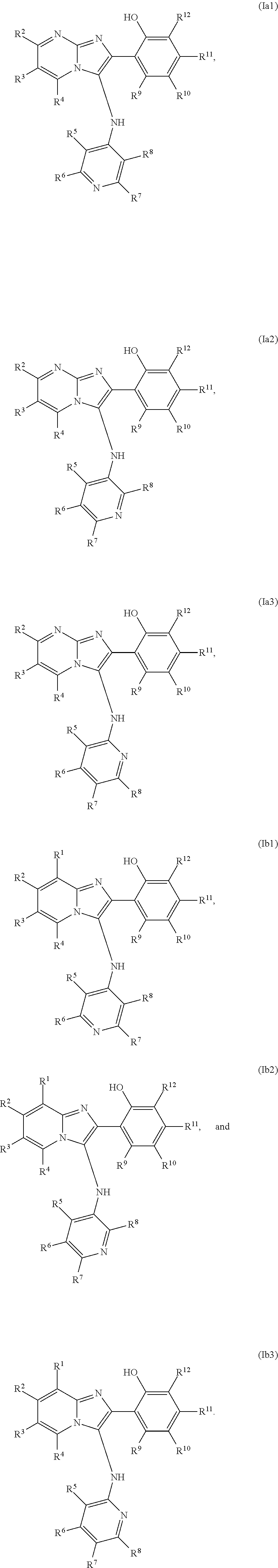

12. (canceled)

13. The compound of claim 1, which is:

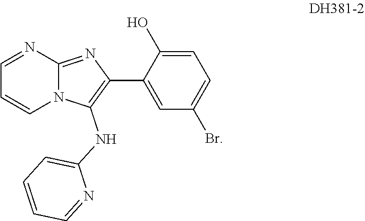 [SMILES: Oc1ccc(Br)cc1-c1nc2ncccn2c1Nc1ccccn1]

14. A compound of formula (II): 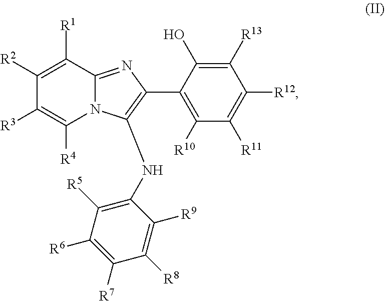
wherein:
R¹, R², R³, R⁴, R⁶, R⁷, R⁸, R¹⁰, R¹¹, R¹², and R¹³ are independently selected from the group consisting of H, CH₃, CH₂CH₃, CH₂CH₂CH₃, CH(CH₃)₂, F, Cl, Br, and I,
R⁵ and R⁹ are independently selected from the group consisting of CH₃, CH₂CH₃, CH₂CH₂CH₃, CH(CH₃)₂, F, Cl, Br, and I;
or a salt, solvate, tautomer, or stereoisomer thereof.

15. The compound of claim 14, wherein at least one of the following applies:
R¹¹ is Br;
R⁶, R⁷, and R⁸ are H.

16. (canceled)

17. The compound of claim 14, which is selected from the group consisting of:

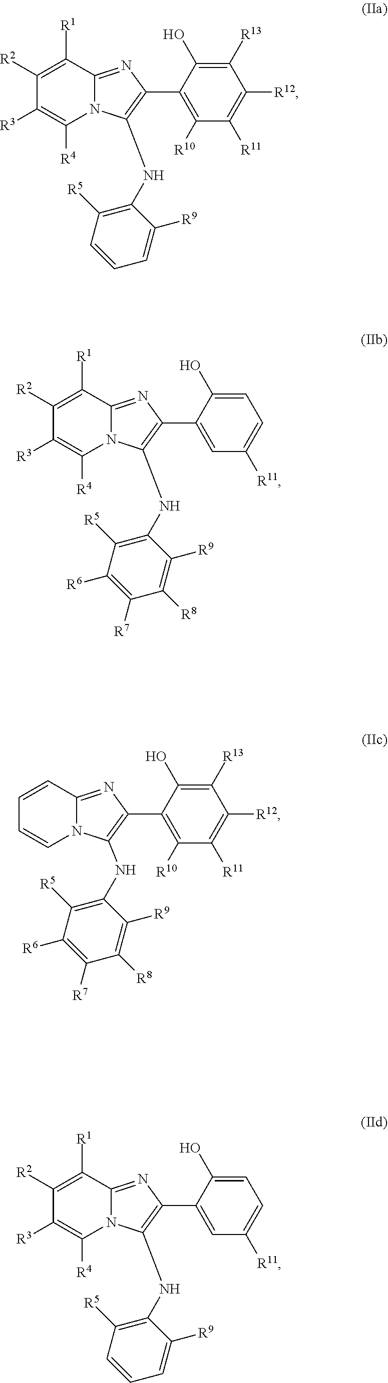

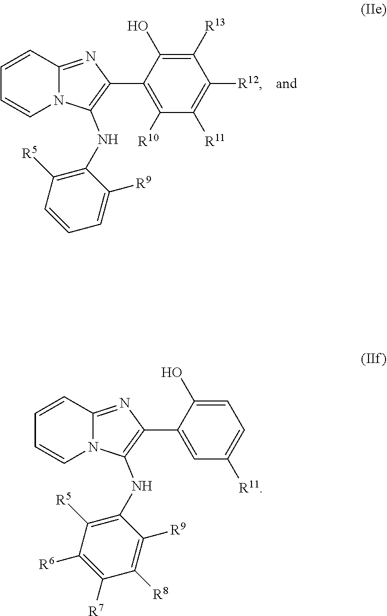

18. (canceled)

19. The compound of claim 14, which is:

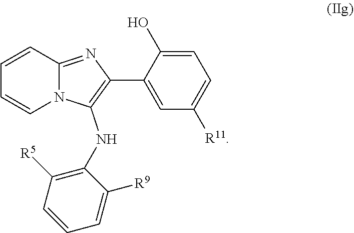

20. The compound of claim 14, which is:

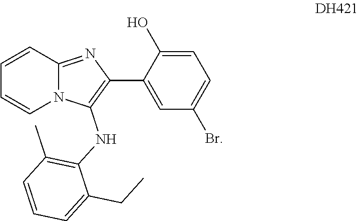 [SMILES: CCc1cccc(C)c1Nc1c(-c2cc(Br)ccc2O)nc2ccccn12]

21. A method of treating, ameliorating, or preventing retinal degeneration in a subject, the method comprising administering to the subject a therapeutically effective amount of a compound of claim 1.

22. The method of claim 21, wherein the retinal degeneration comprises age-related macular degeneration (AMD), optionally wherein the retinal degeneration comprises ‘dry’ AMD.

23. (canceled)

24. A method of treating, ameliorating, or preventing an anterior segment ocular disorder in a subject, the method comprising administering to the subject a therapeutically effective amount of a compound of claim 1.

25. The method of claim 24, wherein the disorder comprises at least one selected from the group consisting of Fuchs Endothelial Corneal Dystrophy, cataracts, glaucoma, and keratoconus.

26. A method of treating, ameliorating, or preventing cell death, and/or promoting cell viability, in a subject, the method comprising administering to the subject a therapeutically effective amount of a compound of claim 1.

27. The method of claim 26, wherein at least one of the following applies:
the cell death comprises oxidative stress-induced cell death;
the cell comprises a lens epithelial cell;
the cell death is associated with at least one disease selected from the group consisting of heart failure and other cardiovascular; pulmonary fibrosis, kidney disease, diabetic macular edema and retinopathies, neurodegeneration, mitochondrial myopathy, Barth's syndrome, and liver disease.

28. (canceled)

29. (canceled)

30. A method of treating, ameliorating, or preventing blue light damage in a subject's lens epithelial cell, the method comprising administering to the subject a therapeutically effective amount of a compound of claim 1.

31. The method of claim 21, wherein at least one of the following applies:
the compound is formulated in a pharmaceutically acceptable composition further comprising at least one pharmaceutically acceptable excipient;
the compound is administered ocularly;
the compound is one of the following:

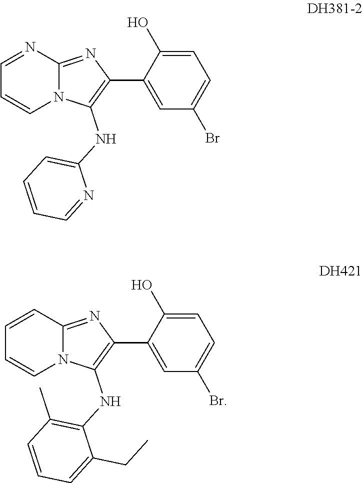 [SMILES: Oc1ccc(Br)cc1-c1nc2ncccn2c1Nc1ccccn1 ; CCc1cccc(C)c1Nc1c(-c2cc(Br)ccc2O)nc2ccccn12]

32. (canceled)

33. (canceled)

34. A pharmaceutical composition comprising a compound of claim 1 and at least one pharmaceutically acceptable excipient.

35. The pharmaceutical composition of claim 34, wherein at least one of the following applies:
the pharmaceutical composition is formulated for ocular administration;
the pharmaceutical composition comprises gamma cyclodextrin (γ-cyclodextrin);
the pharmaceutical composition has pH of about 5-8;
the pharmaceutical composition is lyophilized;
the pharmaceutical composition further comprises a thickening agent.

36-39. (canceled)
